(19) United States
(12) Patent Application Publication
(10) Pub. No.: US 2006/0069350 A1
(43) Pub. Date: Mar. 30, 2006

(54) Medical syringe injector pen

(76) Inventors: David R. Buenger, Roswell, GA (US); Carl E. Griffin, Marietta, GA (US)

Correspondence Address: GARDNER GROFF SANTOS & GREENWALD, P.C., 2018 POWERS FERRY ROAD, SUITE 800, ATLANTA, GA, 30339, US

(21) Appl. No.: 10/954,523

(22) Filed: Sep. 30, 2004

(51) Int. Cl. A61M 5/20; A61M 5/32

(52) U.S. Cl. 604/135; 604/136; 604/197

(57) ABSTRACT

An apparatus for holding a syringe and injecting fluid from the syringe. The apparatus includes a barrel having a bore extending therethrough for receiving a body portion of the syringe, a drive spring, and a telescoping piston movable under the influence of the drive spring to advance the syringe from a retracted position within the barrel to an advanced position wherein a needle portion of the syringe extends outwardly of the barrel, and to compress a plunger portion of the syringe relative to the body portion of the syringe to inject the fluid through the needle portion. After the fluid is injected, the needle is retracted back into a shielded position out of view from the user.

### TECHNICAL FIELD

The present invention relates generally to the field of medical injection aids for pharmaceutical delivery, and in particular to an injector pen for housing a syringe and delivering an injection to a user.

### BACKGROUND OF THE INVENTION

For those persons who require an injection of a pharmaceutical agent, such as insulin, allergy serum, or any number of vaccines, but fear being stuck with a needle, the injection process can be a stressful and painful experience. This ordeal can be exacerbated for those persons who require daily injections and/or who must frequently administer their own injections, such as diabetic persons. Some diabetic persons, such as children and elderly persons, experience difficulty in administering their own injections because of inexperience or lack of dexterity. Still others just simply fear the sight of the sharp needle.

Attempts have been made to provide injection aids, or injector pens as commonly referred to in the industry, that hide the syringe during the injection process. Many of these injector aids resemble the shape and size of a pen. Typically, the user loads a prefilled, disposable syringe into the pen, places the pen against his or her skin at the injection site, and then presses a button to administer the injection.

However, many such injector pens have been found to be inadequate because they are quite cumbersome to use and/or because they lack desirable performance attributes. For example, some injector pens are difficult and time consuming to load with a syringe. And because the sharp needle of a used syringe may project outwardly after the injection is administered from some previously known injector pens, a risk of inadvertent needle-sticks and transmission of dangerous blood-borne pathogens is present. Also, with some injector pens, there is a possibility that the syringe can inadvertently fall out of the pen during loading and unloading, which could cause injury to the person using the pen.

Thus it can be seen that needs exist for an improved injection device, or injector pen, and for methods to facilitate the administering of a subcutaneous injection in a safe and easy manner and to prevent the inadvertent ejection of the syringe. Needs further exist for such mechanisms and methods that are readily adaptable for use with existing disposable syringes and that effectively hide the syringe from plain sight. It is to the provision of an improved injector pen meeting these and other needs that the present invention is primarily directed.

### SUMMARY OF THE INVENTION

In preferred forms, the present invention is an injector pen that accepts fixed and non-fixed needle syringes to facilitate injections for persons who lack the manual and/or visual dexterity to operate standard syringes, or who have fear of syringe needles. The injector pen of the present invention is preferably re-usable with multiple syringes of standard commercial availability. In use, the user preferably does not see the needle during or after injection, to alleviate fear of needles. The device preferably positively engages the syringe to prevent inadvertent removal prior to injection, advances the needle of the syringe to penetrate the skin, fully dispenses the syringe's contents, and retracts the needle out of the skin and back into a shielded position.

In one aspect, the present invention is an apparatus for holding a syringe and injecting fluid from the syringe by use of a drive spring. The device preferably has a barrel with a bore extending throughout the device for receiving a body portion of the syringe. The device preferably has a telescoping piston movable under the influence of the drive spring to advance the syringe from a retracted position within the barrel to an advanced position wherein a needle portion of the syringe extends outwardly of the barrel, and to compress a plunger portion of the syringe relative to the body portion of the syringe to inject the fluid through the needle portion.

In another aspect, the present invention is an apparatus for injecting a pharmaceutical fluid, having a housing for receiving a syringe, a locking mechanism for retaining the syringe in the housing, a drive piston for injecting a pharmaceutical fluid out of the syringe, and an actuator for selectively actuating the drive piston.

In yet another aspect, the present invention is an apparatus for injecting a fluid from a syringe, having a drive spring releasably coupled to a piston for engagement with the syringe to inject the fluid and a return spring for retracting the piston from engagement with the syringe upon release of the piston from the drive spring.

In still another aspect, the present invention is an apparatus for injecting fluid from a syringe, having a housing with first and second portions detachably coupled to one another. The second portion of the housing has a syringe receiver with a rotating collar movable between a first position for engaging the syringe and a second position for releasing the syringe. The first portion of the housing comprises a drive mechanism for actuation of the syringe to inject the fluid.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a perspective view of an injector pen for housing a syringe and injecting a pharmaceutical fluid therein, according to an example embodiment of the present invention.

FIG. 2 shows a receiver portion of the apparatus of FIG. 1 for engaging a syringe therein.

FIG. 3 shows the receiver portion of the apparatus of FIG. 2 with a syringe loaded therein.

FIG. 4 shows the receiver portion of the apparatus of FIG. 2 with a syringe locked therein.

FIG. 5 is a perspective view of a drive portion of the apparatus of FIG. 1 with a portion of its housing removed to show internal components.

FIG. 6 is a cross-sectional view of the apparatus of FIG. 1, having a syringe loaded therein in a charged position.

FIG. 7 is a cross-sectional view of the apparatus of FIG. 1, showing a piston portion partially extended and engaging the syringe therein to advance the syringe.

FIG. 8 is a cross-sectional view of the apparatus of FIG. 1, showing the piston portion fully extended to compress the plunger of the syringe and administer a pharmaceutical fluid.

FIG. 9 is a cross-sectional view of the apparatus of FIG. 1, showing the telescoping piston retracted back from the release plunger, permitting the syringe needle to retract into the barrel of the injector pen.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

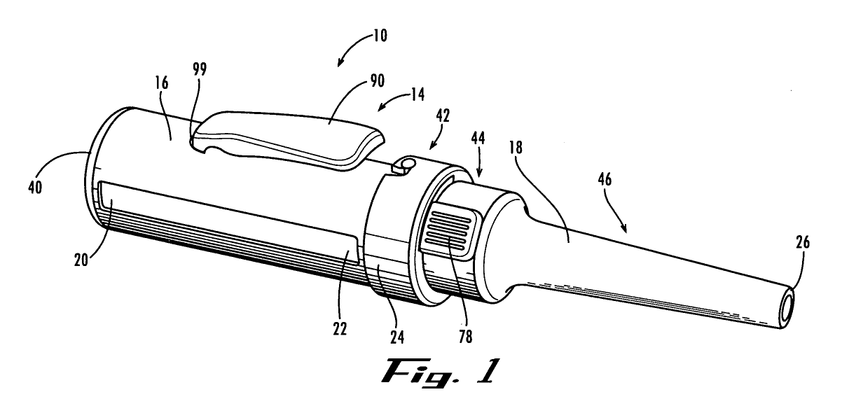

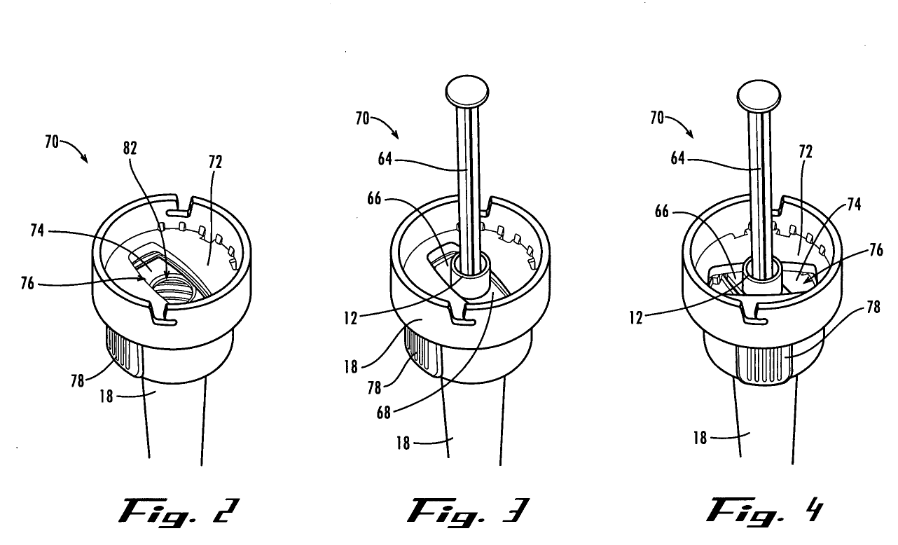

With reference now to FIGS. 1-5, an injection pen 10 suitable for housing a syringe 12 and delivering a pharmaceutical fluid therein to a user is shown in accordance to an example embodiment of the present invention. Preferably, the device is reusable, but alternately, the device can be a single-use device that is discarded after use. The device 10 has a housing 14 including a handle portion 16 and a barrel portion 18 releasably secured to the handle portion. Preferably, the housing 14 is constructed of an opaque material, such as an opaque plastic, that prevents the syringe from being visible to the subject during the injection process.

As depicted in FIG. 1, the overall shape of the housing 14 resembles that of a pen or a screwdriver. The length of the housing is preferably long enough to accommodate a standard, disposable or re-usable syringe, without having any portion of the syringe extending outwardly of the housing. Thus, once loaded into the device, the syringe 12 is wholly contained within the housing 14 and cannot be seen through the housing 14.

The handle portion 16 has a first or proximal end 20 and a second or distal end 22, and the barrel portion 18 has a first or proximal end 24 and a second or distal end 26 defining a needle opening. The distal end 22 of the handle portion 16 adjoins with the proximal end 24 of the barrel portion 18. Preferably, the handle portion 16 has pair of opposing studs 30 and 32 at its distal end 22, which cooperate with a pair of J-shaped (or inverted J-shaped) slots 34 and 36 in the proximal end 24 of the barrel portion 18 of the housing 14. Thus, the handle and barrel portions of the housing 14 can be releasably secured to one another in the manner of a bayonet coupling, by aligning the studs 30 and 32 with the slots 34 and 36, and rotating the handle portion 16 of the housing 14 until the studs lock in place in the slots. Alternately, other fastening methods, including but not limited to, snap connections and threaded connections, can be employed as well.

Preferably the handle portion 16 of the housing 14 is generally cylindrical or prismatic in shape, and has a cap 40 permanently affixed at its proximal end 20. The barrel portion 18 is preferably also generally cylindrical or prismatic in shape, comprising an elongate hollow tubular member having an internal bore extending lengthwise therethrough. In the depicted embodiment, the barrel portion 18 is a continuous piece having three tiered sections, a first section 42, a second section 44, and a third section 46. The inner diameter of the first section 42 is approximately equal to or slightly larger than the outer diameter of the handle portion 16 of the housing 14 so that the two portions can readily couple together. The outer diameter of the first section 42 is larger than the diameter of the second section 44, which is larger than the diameter of the third section 46, to define a stepped external housing configuration. In alternate embodiments, a smoothly tapered configuration is provided. The inner bore diameter of the third section 46 is preferably slightly larger than the outer diameter of the syringe 12, to allow the syringe 12 to smoothly advance through the bore toward the injection site upon actuation, without impeding the syringe's movement. Thus, a first annular ledge or shoulder 48 is formed where the first section 42 and the second section 44 meet, and a second annular ledge 50 is formed where the second section 44 and the third section 46 meet. At least one, and preferably two, opposing circumferential slots preferably extend through the annular ledge 48 for actuating a syringe-locking mechanism as described below.

As seen more clearly in FIGS. 2-6, a typical syringe 12 suitable for use with the injector pen apparatus of the present invention has a generally cylindrical body 60 defining a reservoir for containing a quantity of pharmaceutical fluid therein. One end of the syringe 12 is open for receiving a plunger 62 that slides within the cylindrical body 60 for intake and delivery of the fluid. A needle 64 extends from the other end of the body 60 for penetrating the subject's skin and delivering a subcutaneous injection. Thus, the plunger 62, when pushed inwardly, forces the pharmaceutical fluid through the needle 64. Also, the syringe 12 preferably has a set of finger stops or flanges 66 and 68, on opposing sides of the body 60, near the open end of the body 60. The finger stops 66 and 68 typically assist the user in drawing the pharmaceutical fluid into the syringe 12 and dispensing the pharmaceutical fluid into the user by allowing the user to rest his or her middle and index fingers against the stops 66 and 68, while using his or her thumb to force the plunger 62 into the cylindrical body 64. The injector pen of the present invention is preferably adaptable for use with one or more types of standard, commercially-available syringes.

Referring particularly to FIGS. 24 and FIG. 6, the injection apparatus 10 preferably includes a syringe locking mechanism 70 for receiving and locking the syringe 12 in place. The locking mechanism 70 is preferably located within the first and second tiered sections 42 and 44 of the barrel 18 of the housing 14, and comprises a rotatable collar member 72 and a syringe abutment flange 74 that is initially positioned adjacent and forward of the rotatable collar member 72.

The rotatable collar member 72 preferably has an elongate opening 76 therein that is of similar shape but slightly larger than the profile of the finger stops 66, 68 of the syringe 12 to be used with the device. The rotatable collar member 72 preferably also has at least one tab 78, and preferably a second tab 80, that extend through the circumferential slots of the first annular ledge 48. The tabs 78 and 80 can be rotated along the outer surface of the second tiered section 44 of the barrel 18 of the housing 14. When tabs 78 and 80 are rotated, the collar member 72 rotates, preferably, through about 90°, to capture the finger stops 66, 68 of the syringe. The syringe abutment flange 74 preferably has a circular passage 82 therethrough for receiving the cylindrical body 60 of the syringe 12 and a recessed area 84 for receiving the finger flanges 66, 68 such that the finger flanges rest within the recessed area and do not rotate with the collar 72. A syringe retraction spring or other biasing element 86, acting in compression, biases the syringe abutment flange 74 towards the rotatable collar member 72. One end of the biasing element 86 preferably abuts the second annular ledge 50 and the second end abuts the front surface of the syringe abutment flange 74, and biases the syringe abutment flange 74 towards the rotatable collar member 72. The syringe 12 is loaded into the locking mechanism 70 by inserting the body of the syringe through the elongate opening 76 of the rotatable collar member 72 and into the circular passage 82 of the syringe abutment flange 74. Preferably, the needle 64 of the syringe 12 does not contact the walls of the housing 14 of the device 10 when loading, so as not to contaminate the sterile needle. The finger stops 66 and 68 of the syringe 12 pass through the elongate opening 76 and rest in the recessed area 84 of the syringe abutment flange 74, which acts as a mechanical stop for the syringe 12. Once the syringe 12 is loaded, the tabs 78 and 80 are rotated one-fourth of a turn (about 90 ), which in turn rotates the collar member 72, moving the elongate slot 76 out of alignment with the finger stops 66, 68 of the syringe 12, thereby locking the syringe in place and preventing its removal from the device 10 during assembly and disassembly.

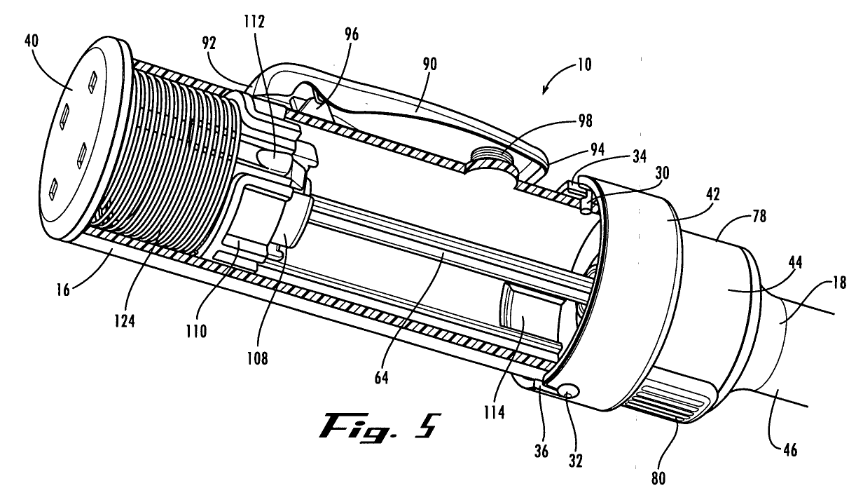

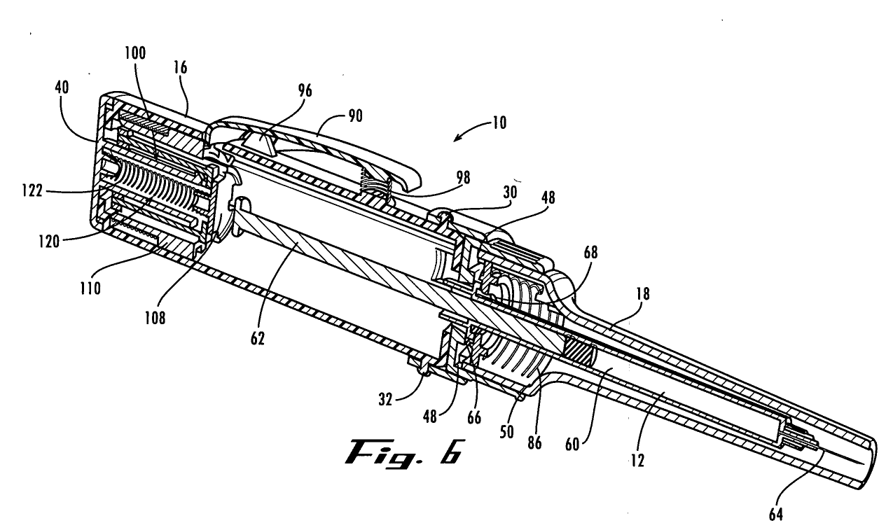

With reference now to FIGS. 5-9, the injection apparatus 10 preferably also includes a drive mechanism and an actuation member or trigger 90 on the handle portion 16 of the housing 14 for releasing the drive mechanism and actuating the device to deliver the pharmaceutical fluid from the syringe 12 to the user. In the depicted embodiment, the trigger 90 is a lever arm that extends from a first end 92 near the proximal end 20 of the handle portion 16 to a second end 94 near the distal end 22 of the handle. Near the first end 92 of the lever 90 is a fulcrum 96. A trigger spring or other biasing element 98 biases the second end 94 outwardly from the device 10, which in turn biases the first end 92 of the lever 90 inwardly. The first end 92 of the lever 90 is biased inwardly through an opening 99 in the housing 14 so that the lever 90 engages a telescoping drive piston 100 located within the handle portion 16 of the housing 14, and retains the drive piston in a retracted position (FIG. 6) when the device 10 is in its charged or loaded position. When the trigger 90 is actuated, the telescoping piston is released to advance the syringe needle through the subject's skin, and to compress the plunger to inject the contents of the syringe.

Preferably, the telescoping piston 100 has a plurality of extensibly engaged segments (four segments 102, 104, 106, and 108 are shown). A release plunger 110 engages the distal-most extending and outermost segment of the telescoping piston to drive the extension of the telescoping piston. The distal-most extending segment 108 of the telescoping piston 100 preferably has a front contact surface for engaging the back of the plunger 62 of the syringe 12 so as to advance the syringe and compress the plunger of the syringe to carry out the injection. The release plunger 110 is preferably releasably engaged with the distal-most extending segment 108 of the telescoping piston 100, and a drive spring 124 is preferably provided in compression between the cap 40 of the handle 16 and the release plunger, to drive the release plunger (and the telescoping piston carried therewith) from the retracted position shown in FIG. 6, to advance the syringe and inject its contents.

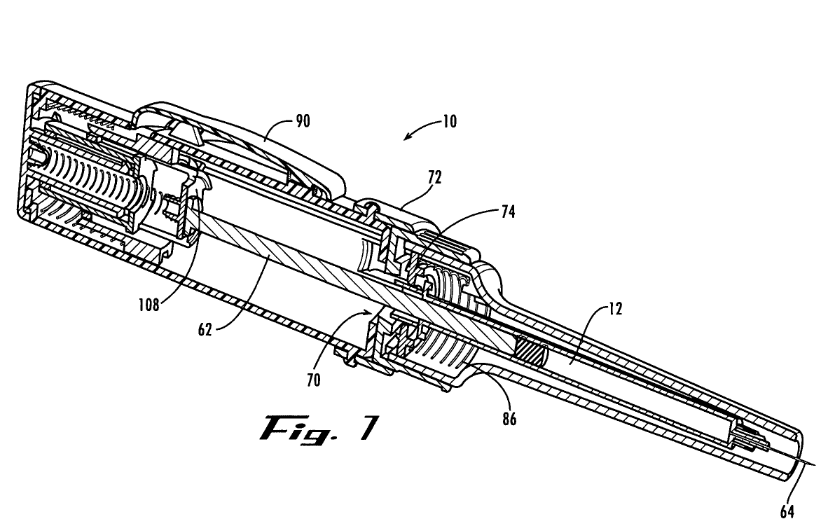

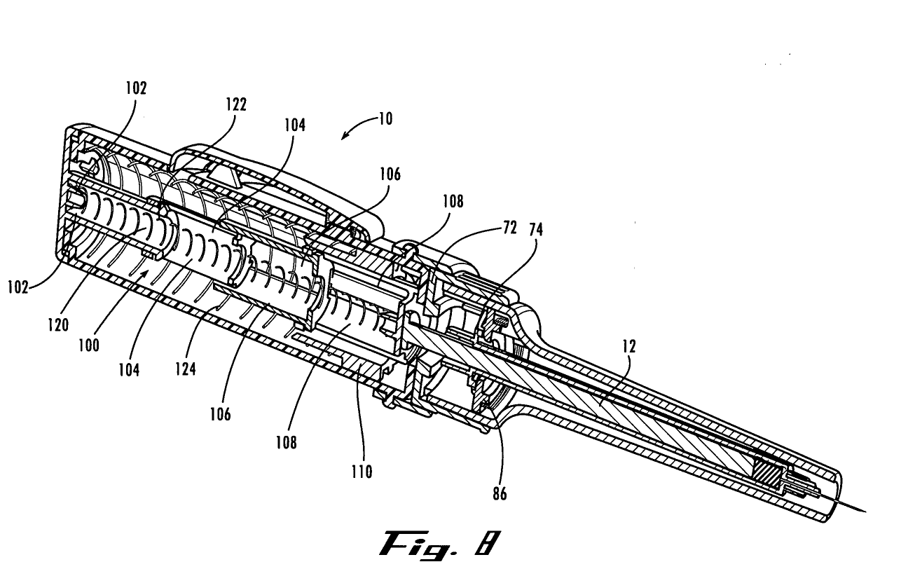

The force required to overcome the rearward bias of the syringe retraction spring 86 and thereby advance the syringe 12 through the barrel 18 is preferably less than the force required to compress the plunger of the syringe and dispense the syringe's contents. As a result, as the telescoping piston 100 extends under the influence of drive spring 124 into contact with the syringe 12, the syringe is first advanced to drive the needle 64 of the syringe outwardly through the opening in the end of the barrel and into the subject's skin (FIG. 7). Continued extension of the telescoping piston then compresses the plunger of the syringe and injects the syringe's contents.

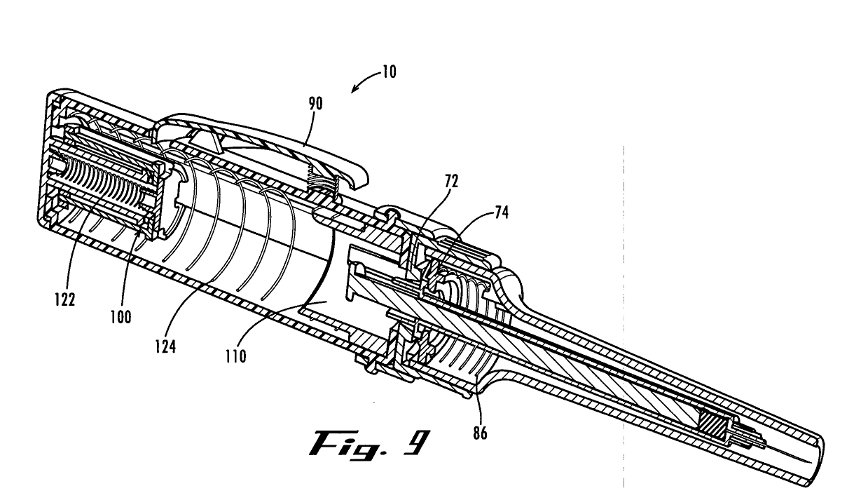

Upon reaching its fully extended position wherein the contents of the syringe 12 have been fully dispensed, as shown in FIG. 8, the telescoping piston 100 is preferably released from engagement with the release plunger 110, and the syringe needle 64 is withdrawn back into the barrel 18 under the bias of the syringe retraction spring 86, out of contact with the subject's skin and into a retracted position to prevent inadvertent contact with the contaminated sharp needle. As seen more clearly in FIG. 5, the release plunger 110 has a cantilevered protrusion 112 that is compressed into engagement with the distal-most extending segment of the telescoping piston 100 as the piston is extended through the handle 16, and that moves into a cutout or recess 114 in the interior surface of the handle 16 near its distal end 22, when the telescoping piston 100 reaches its fully extended position, to release the telescoping piston from engagement with the release plunger.

A hollow channel 120 within the telescoping piston 100 preferably houses a piston retraction spring 122 or other biasing element, which operates in tension to bias the telescoping piston 100 back toward a retracted position after release from the release plunger 110. One end of the biasing element 122 preferably engages the cap 40 and the other end engages an inner surface of the distal-most extending segment 108 of the telescoping piston 100. The biasing element 122 collapses the intermediate segments 102, 104, and 106 of the telescoping piston 100 in a direction generally towards the proximal end 20 or cap 40 of the device 10. The syringe retraction spring 86 is then free to expand, driving the abutment flange 74 and the spent syringe 12 rearward, retracting the sharp tip of the syringe's needle 64 from the subject's skin and back into the barrel 18, where it is shielded from inadvertent contact, as shown in FIG. 9.

In operation, the user separates the handle portion 16 of the housing 14 from the barrel portion 18 and then loads a syringe 12 prefilled with a pharmaceutical fluid into the locking mechanism 70 of the device 10. The rotatable collar member 72 is rotated to lock the syringe 12 into the barrel portion 18 of the housing 14. If the drive mechanism is not energized, the distal end 26 of the barrel 18 can be pressed into the handle 16 to compress the piston 100 and drive spring 124, and to engage the trigger 90. Alternatively, a cocking mechanism is provided for compressing the piston and drive spring and engaging the trigger. Once the syringe 12 has been loaded, the user reconnects the handle portion 16 of the housing 14 to the barrel portion 18. The user then places the open end 26 of the device 10 against his or her skin at the injection site. The user actuates the device 10 by pressing down on the second end 92 of the trigger lever 90, which overcomes the force of the biasing element 98, as depicted in FIG. 6. In turn, the lever 90 releases the telescoping piston 100 under the influence of drive spring 124. The piston 100 and drive spring 124 overcome the rearward bias of spring 86, causing the syringe 12 to advance towards the end 26 of the device to allow needle 64 to penetrate the user's skin. The telescoping piston 100 drives the plunger 62 of the syringe 12 into the body 60 of syringe 12, which injects the pharmaceutical fluid therein to the user. When the piston 100 reaches its fully extended position wherein the syringe's contents have been completely injected, the cantilevered protrusions 112 of the release plunger 110 extend into the cutout or recess 114 of the housing 14, which releases the piston 100 to retract under the influence of piston retraction spring 122. Simultaneously, the advancing bias of the piston 100 on the syringe 12 is relieved, and the syringe retraction spring 86 retracts the syringe abutment flange 74 back to its resting position, which in turn draws the needle 64 back into the housing 14 of the device 10, as depicted in FIG. 9. The injection is now complete, and the user can open the housing to remove and dispose of the syringe 12 by unlocking it from the locking mechanism by rotating tabs 78, 80 in the reverse direction. The device can then be reloaded with a fresh syringe, and the process repeated as needed.

While the invention has been described with reference to preferred and example embodiments, it will be understood by those skilled in the art that a variety of modifications, additions and deletions are within the scope of the invention, as defined by the following claims.

What is claimed is:

1. An apparatus for holding a syringe and injecting fluid from the syringe, said apparatus comprising:
a barrel having a bore extending therethrough for receiving a body portion of the syringe;
a drive spring; and
a telescoping piston movable under the influence of said drive spring to advance the syringe from a retracted position within the barrel to an advanced position wherein a needle portion of the syringe extends outwardly of the barrel, and to compress a plunger portion of the syringe relative to the body portion of the syringe to inject the fluid through the needle portion.

2. The apparatus of claim 1, further comprising a retraction spring for retracting the syringe from the advanced position to the retracted position after injecting the fluid.

3. The apparatus of claim 1, further comprising locking means for retaining the syringe in the barrel.

4. The apparatus of claim 3, wherein the locking means comprises an abutment flange and a rotatable collar for capturing a portion of the body portion of the syringe therebetween.

5. The apparatus of claim 1, further comprising a release plunger driven by the drive spring and releasably coupled to the telescoping piston.

6. The apparatus of claim 5, wherein the release plunger remains coupled to the telescoping piston until the telescoping piston fully compresses the plunger portion of the syringe to inject the fluid, and releases from the telescoping piston after injecting the fluid.

7. The apparatus of claim 6, further comprising a piston retraction spring for retracting the telescoping piston away from the syringe upon release of the telescoping piston from the release plunger.

8. The apparatus of Claim 6, wherein the release plunger comprises at least one cantilevered protrusion that is retained in an inward position in engagement with the telescoping piston by sliding contact along an interior surface of a housing portion of the apparatus, and that flexes outwardly under its own resilience into a recess in the interior surface of the housing to release the telescoping piston.

9. The apparatus of Claim 1, further comprising a handle portion for connection to the barrel to form a housing, and wherein said housing is opaque and fully surrounds the syringe to obscure the syringe from external view during use of the apparatus.

10. The apparatus of Claim 9, wherein the handle portion and the barrel detachably engage one another via a quick-release bayonet coupling.

11. The apparatus of Claim 1, wherein the bore of the barrel accepts fixed-needle and non-fixed-needle syringes.

12. The apparatus of Claim 1, wherein said apparatus is reusable by removing a spent syringe from the barrel after the fluid is injected, and thereafter reloading a fresh syringe.

13. An apparatus for injecting a pharmaceutical fluid, said apparatus comprising:
a housing for receiving a syringe;
a locking mechanism for retaining the syringe in the housing;
a drive piston for injecting a pharmaceutical fluid out of the syringe; and
an actuator for selectively actuating the drive piston.

14. The apparatus of Claim 13, wherein the actuator comprises a lever having a first end that extends through the housing to releasably engage the drive piston when the drive piston is in a charged position.

15. The apparatus of Claim 14, wherein the drive piston comprises a plurality of telescoping segments biased by a drive spring held in compression when the drive piston is in the charged position.

16. The apparatus of Claim 13, wherein the housing is opaque and fully surrounds the syringe to obscure the syringe from external view during use of the apparatus.

17. The apparatus of Claim 13, wherein the locking mechanism comprises an abutment flange and a rotatable collar.

18. The apparatus of Claim 13, wherein the housing comprises first and second portions and a quick-release coupling for connecting the first and second portions to one another.

19. An apparatus for injecting a fluid from a syringe, said apparatus comprising:
a drive spring-releasably coupled to a piston for engagement with the syringe to inject the fluid; and
a return spring for retracting the piston from engagement with the syringe upon release of the piston from the drive spring.

20. The apparatus of Claim 19, further comprising a syringe retraction spring for retracting the syringe from an advanced position to a retracted position upon release of the piston from the drive spring.

21. An apparatus for injecting a fluid from a syringe, said apparatus comprising a housing having first and second portions detachably coupled to one another, the second portion of the housing comprising a syringe receiver having a rotating collar movable between a first position for engaging the syringe and a second position for releasing the syringe, and the first portion of the housing comprising a drive mechanism for actuation of the syringe to inject the fluid.
